(19) United States
(12) Patent Application Publication
(10) Pub. No.: US 2011/0158915 A1
(43) Pub. Date: Jun. 30, 2011

(54) NANOSHELLS WITH TARGETED ENHANCEMENT OF MAGNETIC AND OPTICAL IMAGING AND PHOTOTHERMAL THERAPEUTIC RESPONSE

(75) Inventors: Rizia Bardhan, Berkeley, CA (US); Amit Joshi, Houston, TX (US); Nancy J. Halas, Houston, TX (US)

(73) Assignees: WILLIAM MARSH RICE UNIVERSITY, Houston, TX (US); BAYLOR COLLEGE OF MEDICINE, Houston, TX (US)

(21) Appl. No.: 12/916,041

(22) Filed: Oct. 29, 2010

Related U.S. Application Data
(60) Provisional application No. 61/255,946, filed on Oct. 29, 2009.

(51) Int. Cl. A61K 49/18; A61K 9/14; B05D 3/00; A61P 35/00

(52) U.S. Cl. 424/9.32; 424/490; 427/2.12; 427/2.14

(57) ABSTRACT

A particle and a method of manufacturing a particle that includes a complex, a paramagnetic entity, and a silica layer that encapsulates the paramagnetic entity and the complex. The dielectric layer of the particle encapsulates the complex and the paramagnetic entity such that at least a portion of an outer surface of the complex is covered by the paramagnetic entity. In addition, the particle may or may not include a fluorescent entity encapsulated within the dielectric layer. Also, the particle may or may not include a targeting entity covalently bonded to the silica layer.

### CROSS-REFERENCE TO RELATED APPLICATIONS

This application claims priority to U.S. Provisional Application No. 61/255,946, entitled “Nanoshells with Targeted Simultaneous Enhancement of Magnetic and Optical Imaging and Photothermal Therapeutic Response,” filed Oct. 29, 2009, which is hereby incorporated by reference in its entirety.

### STATEMENT REGARDING FEDERALLY SPONSORED RESEARCH OR DEVELOPMENT

This invention was made with government support under grant F49550-06-1-0021 awarded by the Air Force Office of Scientific Research and grant W911NF-04-01-0203 awarded by the Department of Defense Multidisciplinary University Research Initiative (MURI). The government has certain rights in the invention.

### BACKGROUND

The development of noninvasive diagnostic imaging modalities such as magnetic resonance imaging (MRI) and fluorescence optical imaging (FOI) is one goal in biomedical research and practice. All imaging techniques in biomedical research and medical practice have their own merits and drawbacks in terms of sensitivity, resolution, data acquisition time, and complexity. While some contrast agents for biological image enhancement have been developed, they are typically limited to the enhancement of a single modality.

### SUMMARY

In general, in one aspect, the invention relates to a particle including a complex and a paramagnetic entity. The particle also includes a dielectric layer that encapsulates the complex and the paramagnetic entity where at least a portion of an outer surface of the complex is covered by the paramagnetic entity. In addition, the particle may or may not include a fluorescent entity encapsulated within the dielectric layer. Also, the particle may or may not include a targeting entity covalently bonded to the dielectric layer.

In general, in one aspect, the invention relates to a method of manufacturing a particle that includes encapsulating a complex and a paramagnetic entity within a dielectric layer, where the paramagnetic entity covers at least a portion of an outer surface of the complex. Also, the method may or may not include incorporating a fluorescent entity into the dielectric layer. In addition, the method may or may not include covalently bonding a targeting entity to the encapsulating dielectric layer.

Other aspects of the invention will be apparent from the following description and the appended claims.

### BRIEF DESCRIPTION OF DRAWINGS

FIGS. 1A-1B show a schematic of the particles in accordance with one or more embodiments of the invention.

FIG. 2 shows a flow chart of a method in accordance with one or more embodiments of the invention.

FIG. 3 shows absorbance spectra in accordance with one or more embodiments of the invention.

FIG. 4 shows x-ray diffraction patterns in accordance with one or more embodiments of the invention.

FIG. 5 shows fluorescence spectra in accordance with one or more embodiments of the invention.

FIGS. 6A-6C show the magnetization of the particle in accordance with one or more embodiments of the invention.

FIGS. 7A-7B show the magnetic resonance image intensity and spin-spin relaxation rate of the particles in accordance with one or more embodiments of the invention.

### DETAILED DESCRIPTION

Specific embodiments of the invention will now be described in detail with reference to the accompanying FIGs. Like elements in the various FIGs. are denoted by like reference numerals for consistency.

In the following detailed description of embodiments of the invention, numerous specific details are set forth in order to provide a more thorough understanding of the invention. However, it will be apparent to one of ordinary skill in the art that the invention may be practiced without these specific details. In other instances, well-known features have not been described in detail to avoid unnecessarily complicating the description.

In general, embodiments of the invention relate to a particle with properties to enhance fluorescence optical imaging and/or magnetic resonance imaging. Further, embodiments of the invention relate to a particle that may enhance multiple imaging technologies simultaneously. Further, embodiments of the invention may combine the aforementioned imaging enhancement with antibody and/or peptide targeting and/or photothermal therapeutic actuation.

One or more embodiments of the invention relate to a particle that may be constructed by coating a complex with a silica epilayer doped with paramagnetic entities and/or fluorescence entities. Also, one or more embodiments of the invention relate to a particle with the aforementioned features and a targeting entity bound to the silica epilayer.

In one or more embodiments of the invention, a complex may refer to a nanoshell. A nanoshell is a substantially spherical dielectric core surrounded by a thin metallic shell. The plasmon resonance of a nanoshell may be determined by the size of the core relative to the thickness of the metallic shell. A complex may also include other core-shell structures, for example, a metallic core with one or more dielectric and/or metallic layers using the same or different metals. For example, a complex may include a gold or silver nanoparticle, spherical or rod-like, coated with a silica layer and further coated with another gold or silver layer. A complex may also include other known nanostructures, for example nanorods, nanotubes, nanocages or hollow metallic shell nanoparticles.

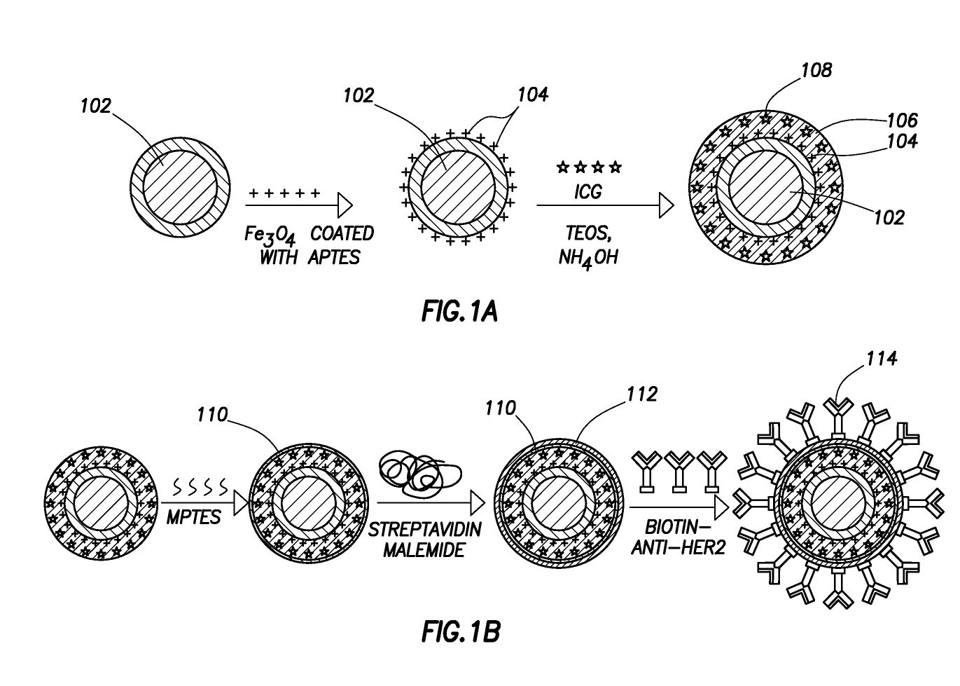

In accordance with one or more embodiments of the invention, a schematic representing the fabrication procedure of the particles is shown in FIGS. 1A and 1B. In FIG. 1A, complex 102 may be fabricated as known in the art. For example, nanoshells may be fabricated according to U.S. Pat. No. 6,685,986, hereby incorporated by reference in its entirety. The relative size of the dielectric core and metallic shell, as well as the optical properties of the core, shell, and medium, determines the plasmon resonance of a nanoshell. Accordingly, the overall size of the nanoshell is dependent on the absorption wavelength desired. For example, to obtain a plasmon resonance in the near infrared region of the spectrum (700 nm-900 nm) a substantially spherical silica core having a diameter between 90 nm-175 nm has a gold metallic layer between 4 nm-35 nm.

A paramagnetic entity 104 may then be fabricated, or obtained, and covalently attached to the surface of the complex 102. Examples of a paramagnetic entity 104 include, but are not limited to, iron oxide, gadolinium chelated agents, or manganese chelated agents. For example, water soluble Fe₃O₄ nanoparticles, from 7 nm-15 nm in diameter may be synthesized by the reduction of the iron ions and functionalized with a molecular linker, for example, (3-aminopropyl) triethoxysilane (APTES). The amine functionalization may facilitate the bonding of the paramagnetic entity to the nanoshell. One of ordinary skill in the art will appreciate that other functional groups may be used to facilitate the bonding between the paramagnetic entity 104 and the complex 102. For example, in the case of paramagnetic nanoparticles, thiol groups, di-amine molecules, and di-thiol molecules may be used. In addition, one of ordinary skill will appreciate that the molecular linker may be chosen based on the specific complex used. For example, a thiol or amine linker may be used for complexes and/or contrast agents that are terminated by a metallic layer, such as nanoshells or nanorods.

The complex 102 may then be coated with the paramagnetic entity 104, for example, amine terminated Fe₃O₄ nanoparticles. The number of paramagnetic entities bonded to the surface of the complex may be influenced by the relative size of the complex to the paramagnetic entity, the relative charges of the complex and paramagnetic entity, and the linker molecule used. The number of paramagnetic entities per complex may determine the overall magnetic properties of the particle and, thus, the magnetic activity of the particle. Those skilled in the art will appreciate that the paramagnetic entities may not be uniformly distributed across the entire surface of the complex or cover the entire surface of the complex.

The complex 102 coated with the paramagnetic entities may then be surrounded with a dielectric layer 106. The dielectric layer 106 may encapsulate, or completely encompass, the paramagnetic entity 104 and the complex 102. Alternatively, the paramagnetic entity may be deposited simultaneously with the dielectric layer. In one or more embodiments, the dielectric layer may be deposited immediately following the deposition of the paramagnetic entity. In one or more embodiments, the linker molecule binding the complex to the paramagnetic entity may or may not be necessary. The thickness of the dielectric layer may contribute to the desired overall size of the particle. For example, silica (SiO₂) may be used as the dielectric layer to encapsulate the paramagnetic entity and the complex. The silica layer may be deposited by the condensation of tetra-ethyl ortho-silicate in chemically basic environment. The relative concentration of the reactants may determine the thickness of the silica layer. The silica layer may be 3 nm-30 nm thick depending on the overall size of the particle desired (in conjunction with the plasmon resonance of the particle and the number and size of paramagnetic entities desired). In addition to silica, other dielectric materials may be used, for example titanium dioxide, or other polymer-based dielectrics, such as polyvinyl including polymers may be used.

The dielectric layer 106 may include a fluorescent entity 108. In one or more embodiments, a molecular fluorophore, for example indocyanine green (ICG), may be incorporated within the silica layer 106. The fluorescent entity 108 may be incorporated into the dielectric layer 106 during the deposition of the dielectric layer 108. The specific fluorescent entity used may be chosen based on the absorption/emission of the fluorescent entity 108 relative to the plasmon resonance of the complex 102 to allow the complex 102 to enhance the fluorescence response of the fluorescent entity 108. The fluorescent entity 108 may also be chosen based on the environment and wavelengths of any subsequent measurements made using the particle.

The fluorescent entity 108 may be incorporated into the silica layer with the aide of an additional chemical linker. The chemical linker may or may not be chemically bonded with the fluorescent entity. For example, in the case where the fluorescent entity 108 is ICG and the dielectric layer 106 is silica, the ICG may be dispersed in a solution of APTES to help facilitate the incorporation of the fluorescent entity 108 into the dielectric layer 106.

The dielectric layer 106 may not only trap the fluorescent entity 108, but may also encapsulate the paramagnetic entity 104 and, thus, provide a chemically inert and biocompatible surface. The encapsulation of the fluorescent entity 108 may also contribute to the fluorescent properties of the fluorescent entity 108. In a specific example, ICG may be stabilized within the protective silica shell, which may decrease any photobleaching of the fluorophore due to interaction with an aqueous media. In addition, the protective silica shell may also allow the straightforward conjugation of antibodies and other biomolecules to the particle for biomedical applications. Those skilled in the art will appreciate the fluorescent entities may not be uniformly distributed across the entire surface of the complex or cover the entire surface of the complex.

FIG. 1B is a schematic of the functionalization of a targeting entity in accordance with one or more embodiments disclosed herein. The surface of the dielectric layer 108 may be terminated with a molecular linker 110 and 112 for linking the surface of the dielectric layer 108 to a specific targeting entity 114. Examples of targeting entities include, but are not limited to, antibodies, aptamers, or peptides. In one or more embodiments of the invention, the buffers used throughout the manufacturing of the particles are sodium phosphate monobasic based buffers with the pH adjusted by the addition of hydrochloric acid and sodium hydroxide.

For example, a silica dielectric layer may be functionalized with thiol groups using a thiolated silane coupling agent 110, such as 3(mercaptopropyl) triethoxysilane. The coupling agent 110 may then be covalently bonded to another molecular linker 112, for example streptavidin maleimide. The maleimide group may form a thioester bond with the thiol on the silica surface. Then, the targeting entity 114 may be bound to the molecular linker 112. For example, Anti-HER2 antibodies may be biotinylated and then bound to the streptavidin conjugated particles at physiological pH and 4° C. In this example, the targeting entity utilizes the extraordinary affinity of avidin for biotin, (Ka=1015 M⁻¹) possibly the strongest known noncovalent interaction of a protein and ligand. One of ordinary skill in the art will appreciate that a biotin/streptavidin system is not the only means of attaching a targeting entity 114 to a dielectric outer layer 106 of a particle. For example, polyethylene glycol based molecules, dentrimers, or thiol-functionalized targeting moieties may be used.

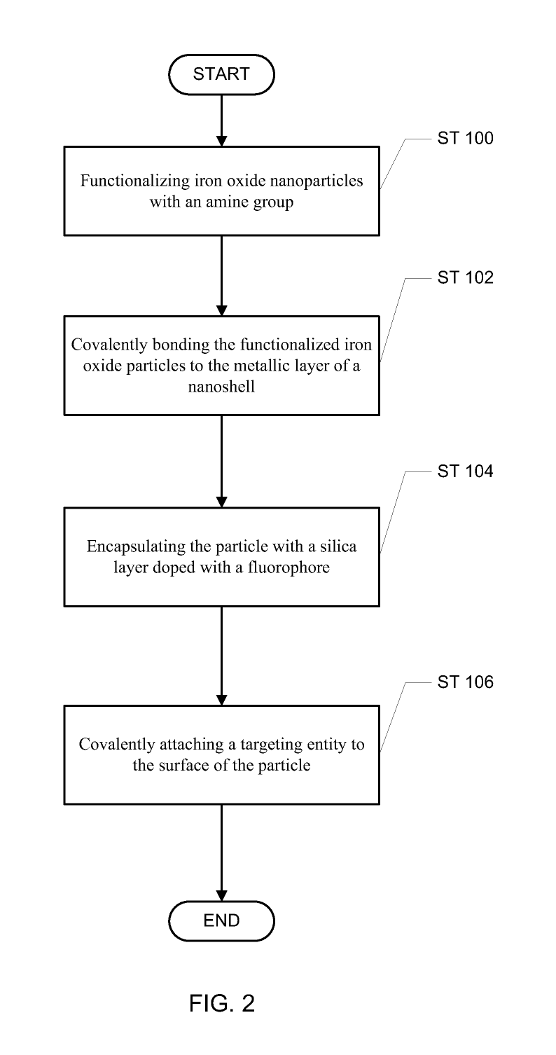

FIG. 2 is a flow chart of a method of manufacturing the particles in accordance with one or more embodiments of the invention. In ST100, the paramagnetic entity (e.g., iron oxide particles) is functionalized with a linker molecule, for example a molecule including an amine group, such as APTES. In ST102, the amine functionalized iron oxide particles are covalently bonded via the linker molecule to the metallic layer of a complex, such as a nanoshell. In ST104, the complex with the paramagnetic entities is encapsulated with a dielectric layer, such as silica. In addition, the dielectric layer may or may not include a fluorescent entity, such as a molecular fluorophore. The fluorophore may be incorporated into the encapsulated dielectric layer during the deposition of the dielectric layer. In ST106, a targeting entity may be attached to the encapsulating dielectric layer, such as an antibody, aptamer, or peptide.

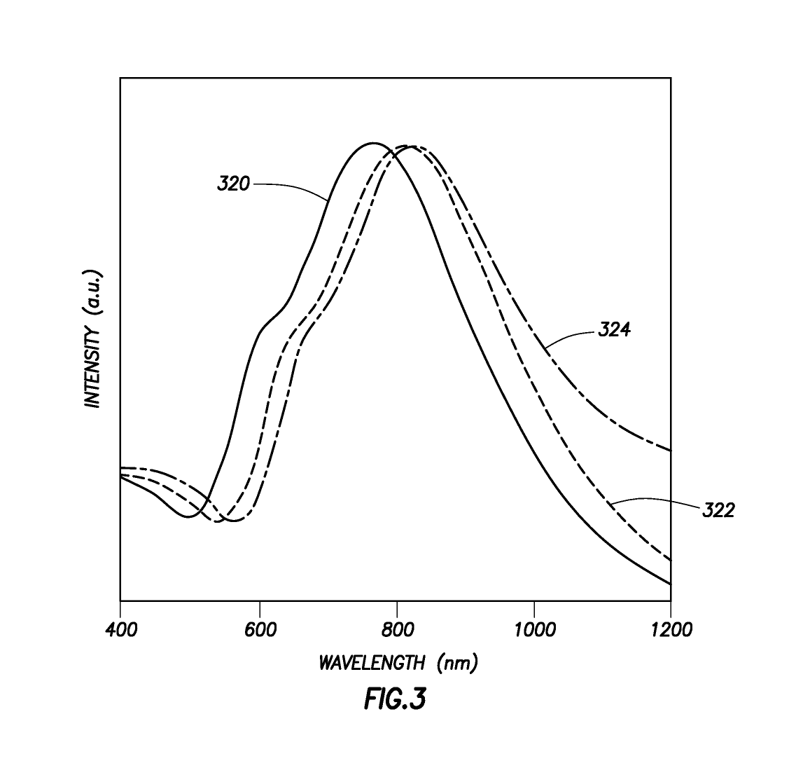

FIG. 3 shows extinction spectra of complexes in accordance with one or more embodiments of the invention. More specifically, FIG. 3 shows the extinction spectra of the nanoshell 320, the complex bonded with the paramagnetic entity 322, and the fluorophore doped encapsulated nanoshell bonded with the paramagnetic entity 324 (hereafter “particle”). The plasmon resonances of the particle 324 may be tuned to match the emission wavelength of the fluorophore to maximize the fluorescence enhancement. The nanoshell 320 may have a plasmon resonance peak at 770 nm, which may redshift to 815 nm when coated with Fe₃O₄ (see 322). The redshift may be due to the higher refractive index of Fe₃O₄ (n=3) relative to the surrounding medium H₂O (n=1.33). The extinction spectrum may shift to 822 nm when the nanoshell bonded with the paramagnetic entity is coated with the encapsulating silica layer 324.

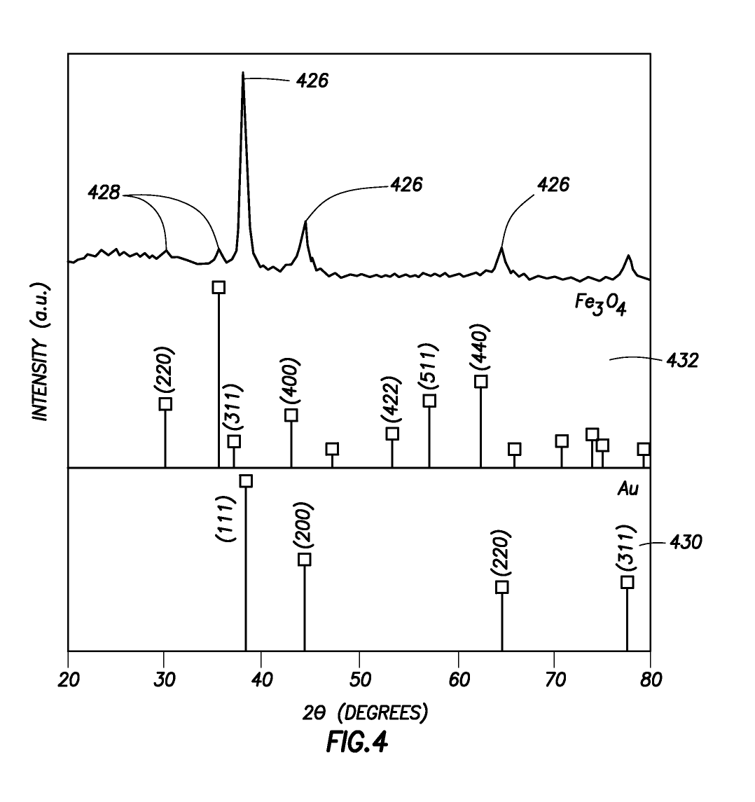

Crystallographic studies using powder X-ray diffraction (XRD) of the particles manufactured in accordance with one or more embodiments is shown in FIG. 4. The XRD shows strong gold peaks 426 as well as Fe₃O₄ peaks 428. The diffraction from gold 426 may dominate the pattern and the Fe₃O₄ peaks 428 may be relatively weaker, due to the heavy atom effect of gold. The gold peaks 426 may represent a cubic phase with cell parameters a=c=4.0786 Å and space group Fm3m (225) (JCPDS card no. 98-000-0230). The Fe₃O₄ peaks 428 observed in the XRD spectrum may indicate a highly crystalline cubic phase of Fe₃O₄ with cell parameters a=c=8.3969 Å and space group Fd-3m (227) (JCPDS card no. 98-000-0294). The corresponding XRD intensity profile of Gold 430 and Fe₃O₄ 432 from the powder diffraction database is included in FIG. 4 for reference.

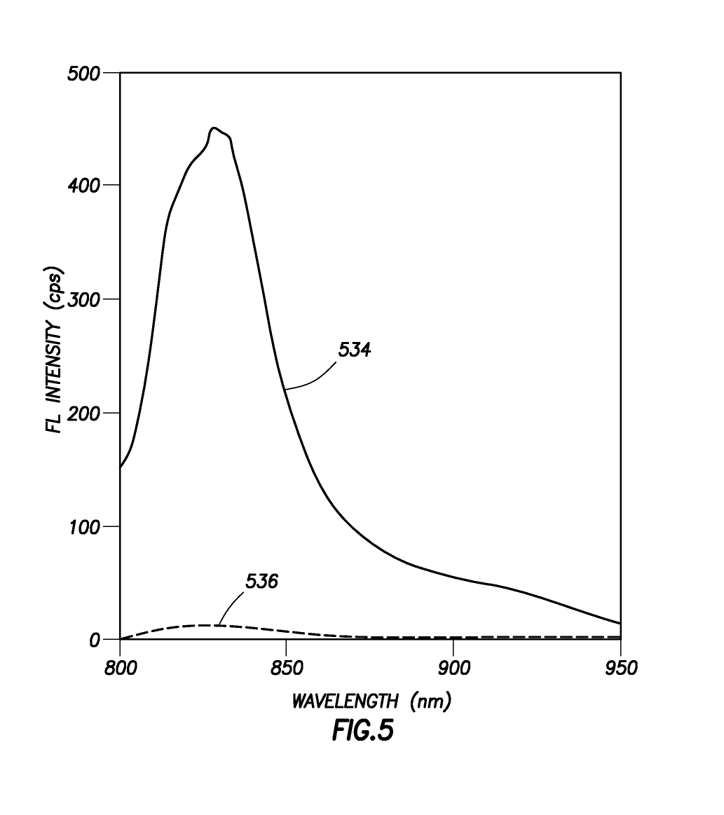

As stated previously, the encapsulating dielectric layer may or may not include a fluorescent entity. Examples of a fluorescent entity include, but are not limited to, molecular visible and near infrared dyes, for example Cy3, Cy5, fluorescein, ICG, green fluorescence protein (GFP), or commercial IR800CW dyes available from LI-COR Biosciences, Lincoln, Nebr. In addition, the fluorescent entity may also be non-molecular in nature, for example quantum dots. FIG. 5 shows an emission spectrum of a particle where the silica layer is doped with the fluorescent molecule ICG in accordance with one or more embodiments of the invention. The fluorescence of the particle (i.e., a complex in which the silica layer is doped with ICG) 534 has a maximum at ˜820 nm associated with the ICG. Also shown in FIG. 5, is the fluorescence of ICG doped within a 180 nm diameter silica nanosphere 536. Silica nanospheres doped with ICG were used as a reference sample rather than ICG in aqueous solution, to ensure the molecules are in similar chemical environments for fluorescence quantification. The fluorescence spectra were collected in solution under identical excitation and detection conditions, to allow for the direct comparison of the particles with a reference sample. Additionally, excess ICG dye was removed by centrifuging both the particle 534 and ICG doped silica reference 536, and the supernatant was monitored to quantify any concentration of fluorophore that may have been present. A maximum fluorescence enhancement of ˜45× is achieved for ˜500±50 nM ICG doped within the silica layer of the particles 534 relative to the reference sample. The enhancement of fluorophore may be primarily attributed to the complex (in this case implemented as a nanoshell).

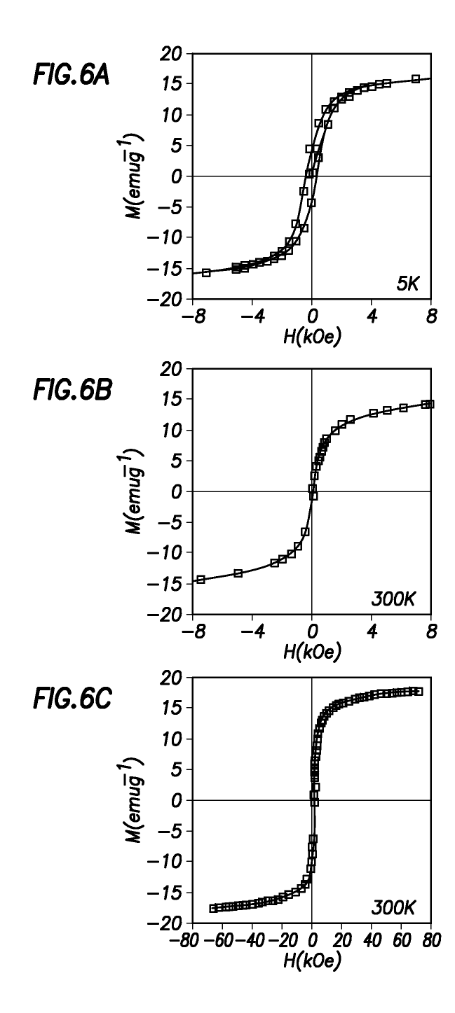

Referring now to FIG. 6A-6C, the magnetization as a function of applied magnetic field at 5 K and 300 K in accordance with one or more embodiments is shown. In FIG. 6A, the magnetization of iron oxide nanoparticles at 5 K demonstrates that the thermal energy may be insufficient to induce magnetic moment randomization. Therefore, the particles may show typical ferromagnetic hysteresis loops with a remanence of 4.2 emug⁻¹ and a coercivity of 385±10.2 Oe. However, at 300K, shown in FIG. 6B, the thermal energy is enough to randomize the magnetic moments or the iron oxide nanoparticles, leading to a decrease in magnetization, thus the nanoparticles show no remanence or coercivity. To evaluate the response of the particles to an external magnetic field in accordance with one or more embodiments disclosed herein, the magnetization was measured at 300 K by cycling the field between −70 kOe and 70 kOe as shown in FIG. 6C. In FIG. 6C, the saturation magnetization, pat, was determined to be 17.98 emug⁻¹ at 70 kOe.

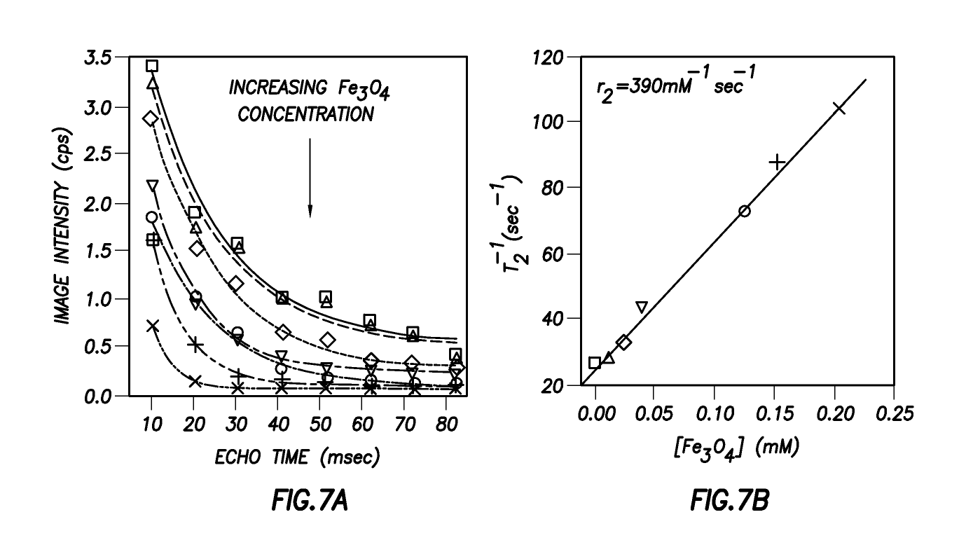

Magnetic Resonance (MR) images of the particles may also be obtained. From the MR images the value of the transverse, or spin-spin relaxation, (T2) may be evaluated as demonstrated in FIGS. 7A and 7B. The T2-weighted MR images (echo time=20 msec) of the particles in aqueous media with Fe₃O₄ concentrations ranging from 0 mM-0.2 mM may be obtained. The Fe₃O₄ concentrations in the particles may be determined by inductively coupled plasma optical emission spectrometry (ICPOES). As the Fe₃O₄ concentration increases, as indicated by the arrow in FIG. 7A, the signal intensity of the MR images may decrease, as expected for T2 contrast agents. T2 may be determined as shown in FIG. 7B from the slope of the normalized image intensity as a function of echo time shown in FIG. 7A. The increasing Fe₃O₄ concentration may lead to a significant decrease in image intensity due to a shortening of the spin-spin relaxation time of water. The specific relaxivity, r₂, which is a measure of the change in spin-spin relaxation rate (T2⁻¹) per unit concentration, is shown in FIG. 7B as 390 mM⁻¹sec⁻¹ for one or more embodiments of the particle. This high r₂ may be due to the large external magnetic field (9.4 T) applied to the particles, as well as the particles magnetic properties.

Based on an analysis of SEM images, a nearly saturated coverage of the NS surface with Fe₃O₄ nanoparticles may be achieved. Thus, the interparticle distance between the Fe₃O₄ nanoparticles bound to the nanoshell surface in this example may be small, resulting in an increased magnetic interaction among the nanoparticles and an enhanced specific relaxivity. Additionally, the porous silica shell present on the particles may increase the molecular motion of any water within the pores and enhance the proton relaxation rate. The aforementioned reasons may result in increased T2 shortening and a consequent increase in specific relaxivity.

Embodiments of the invention may expand the capabilities of particle structures to perform multiple parallel tasks. Embodiments of the invention may allow for noninvasive diagnostic imaging modalities that allow for the integration of targeting, diagnostics, and therapeutics all in one nanoshell based particle. Contrast agents that enhance more than one imaging method may provide a very important advance by enabling the use of multiple modalities to probe the same system. More than one imaging method may yield more information than any single imaging method alone. For example, multimodal contrast agents that simultaneously enhance MRI and FOI may combine the high sensitivity of FOI with the high spatial resolution of MRI. In practice, such a dual-modality contrast agent may be used in a single clinical procedure, for pre- and post-operative MRI, then for intra-operative FOI. As such, one or more embodiments of the invention may provide enhanced imaging before, during, and after a procedure.

Embodiments of the invention may combine the ability to enhance two different imaging technologies simultaneously-fluorescence optical imaging and magnetic resonance imaging—with antibody targeting, and photothermal therapeutic actuation all in the same particle. For example, one or more embodiments of the invention may result in a high T2 relaxivity (390 mM⁻¹sec⁻¹) and 45× fluorescence enhancement using ICG. One or more embodiments of the invention may target HER2+ cells and induce photothermal cell death upon near-IR illumination.

One or more embodiments of the invention may allow for photothermal ablation and FOI at different wavelengths. One or more embodiments of the invention may allow for magneto-ablation using the particle. For example, an applied magnetic field may cause the paramagnetic entity to heat resulting in ablation of a targeted material.

In one or more embodiments of the invention, antibody targeting may be used such that the particle may bind to the surface receptors of specific cell types. In the case of cancer, along with a therapeutic function, such as photothermal heating to induce cell death, the particles may provide a full theranostic spectrum of capabilities in a single, practical particle. The availability of multiple diagnostic and therapeutic modalities in a single particle may streamline the regulatory process in the pharmaceutical drug development pipeline and, thus, may significantly reduce the cost and complexity involved in translating novel therapies from in vitro and in vivo settings to human applications.

One or more embodiments of the invention may allow for the tracking and location of the particle in vivo. For example, MRI or FOI may be used to flow the path of the particles or verify the quantity of the particles at specific locations. Then, the ablation of targeted material may be carried out using an applied optical or magnetic based treatment.

While the invention has been described with respect to a limited number of embodiments, those skilled in the art, having benefit of this disclosure, will appreciate that other embodiments can be devised which do not depart from the scope of the invention as disclosed herein. Accordingly, the scope of the invention should be limited only by the attached claims.

What is claimed is:

1. A particle comprising:
a complex;
a paramagnetic entity; and
a dielectric layer that encapsulates the paramagnetic entity and the complex,
wherein the paramagnetic entity covers at least a portion of an outer surface of the complex.

2. The particle of claim 1, further comprising:
a targeting entity covalently bonded to the dielectric layer.

3. The particle of claim 1, wherein the dielectric layer comprises a fluorescent entity.

4. The particle of claim 3, further comprising:
a targeting entity covalently bonded to the dielectric layer.

5. The particle of claim 4, wherein the targeting entity comprises a linker molecule and an antibody.

6. The particle of claim 3, wherein the fluorescent entity is an indocyanine green (ICG) molecule and the dielectric layer is silica.

7. The particle of claim 1, wherein the complex is a nanoshell.

8. The particle of claim 1, wherein the paramagnetic entity is an iron oxide particle.

9. The particle of claim 8, wherein the iron oxide particle is bonded to the complex via an amine group.

10. The particle of claim 9, wherein the amine group is part of the molecule (3-aminopropyl) triethoxysiline.

11. The particle of claim 8, wherein the iron oxide particle is Fe₃O₄.

12. A method of manufacturing a particle comprising:
encapsulating a complex and a paramagnetic entity with a dielectric layer,
wherein the paramagnetic entity covers at least a portion of an outer surface of the complex.

13. The method of claim 12, further comprising:
incorporating a fluorescent entity into the dielectric layer while encapsulating the particle with the dielectric layer.

14. The method of claim 13, further comprising:
covalently bonding a targeting entity to the dielectric layer.

15. The method of claim 13, wherein the fluorescent entity is a molecule of IR800CW dye and the dielectric layer is silica.

16. The method of claim 12, further comprising:
covalently bonding a targeting entity to the dielectric layer.

17. The method of claim 12, wherein the complex comprises a dielectric core surrounded by a thin metal shell.

18. The method of claim 17, wherein the metal shell is gold.

19. The method of claim 17, wherein the iron oxide particle is bonded to the complex via an amine group, and wherein the amine group is part of the molecule (3-aminopropyl) triethoxysiline.

20. The method of claim 19, wherein the iron oxide particle is Fe₃O₄.
